(19) United States
(12) Patent Application Publication
(10) Pub. No.: US 2026/0066115 A1
(43) Pub. Date: Mar. 5, 2026

(54) Machine Learning Based Emergency Healthcare Information Extraction

(71) Applicant: Cerner Innovation, Inc., Kansas City, MO (US)

(72) Inventor: Praveen Bhat GURPUR, Bengaluru (IN)

(21) Appl. No.: 19/302,235

(22) Filed: Aug. 18, 2025

Related U.S. Application Data
(60) Provisional application No. 63/690,877, filed on Sep. 5, 2024.

(51) Int. Cl. G16H 50/20; G16H 10/60; G16H 50/30

(52) U.S. Cl. CPC G16H 50/20; G16H 10/60; G16H 50/30

(57) ABSTRACT

Embodiments diagnose an emergency room (“ER”) patient. Embodiments receive an identifier of the patient and search and retrieve relevant information factors for the patient from publicly available sources using the identifier using a trained machine learning (“ML”) model. The trained ML model is configured to identify and fetch medically relevant information of the patient in response to the identifier of the patient. Embodiments weight each of the retrieved factors relative to contributing to a diagnoses of the patient and assign a score to each of the retrieved factors and provide the scores and corresponding diagnoses to ER personnel.

### CROSS REFERENCE TO RELATED APPLICATIONS

This application claims priority to U.S. Provisional Patent Application Ser. No. 63/690,877 filed on Sep. 5, 2024, the disclosure of which is hereby incorporated by reference.

### FIELD

One embodiment is directed generally to a computer system, and in particular to a machine learning based computer system for emergency extraction of healthcare information.

### BACKGROUND INFORMATION

In the United States alone there are approximately 130 million emergency room visits per year. Of that number, there are approximately 1 million unconscious or otherwise non-communicative patient emergency room (“ER”) arrivals per year, with about 650,000 John or Mary Doe (i.e., identity unknown) ER admissions. Research studies indicate that there is a better survival rate for those patients when there identity eventually becomes known.

Emergency Medical Technicians (“EMT”s), paramedics and emergency room personnel need quick access to a casualty's pre-existing medical conditions and other vital medical information when the casualty is incapacitated or otherwise (i.e., language, age or dementia) unable to provide it. Conventionally, medic alert bracelets have improved medical outcomes and facilitated patient identification.

### SUMMARY

Embodiments diagnose an emergency room (“ER”) patient. Embodiments receive an identifier of the patient and search and retrieve relevant information factors for the patient from publicly available sources using the identifier using a trained machine learning (“ML”) model. The trained ML model is configured to identify and fetch medically relevant information of the patient in response to the identifier of the patient. Embodiments weight each of the retrieved factors relative to contributing to a diagnoses of the patient and assign a score to each of the retrieved factors and provide the scores and corresponding diagnoses to ER personnel.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments one element may be designed as multiple elements or that multiple elements may be designed as one element. In some embodiments, an element shown as an internal component of another element may be implemented as an external component and vice versa. Further, elements may not be drawn to scale.

FIG. 1 illustrates an example of a system that includes a healthcare information extraction tool/system in accordance to embodiments.

FIG. 2 is a block diagram of the healthcare information extraction system of FIG. 1 in the form of a computer server/system in accordance to an embodiment of the present invention.

FIG. 3 is a flow/block diagram of the functionality of the healthcare information extraction system of FIG. 1 when extracting healthcare/patient information in accordance to embodiments.

FIG. 4 is a flow diagram of the functionality of the healthcare information extraction system of FIG. 1 when extracting medical/healthcare information for an ER patient in accordance to embodiments.

FIGS. 5-8 illustrate an example cloud infrastructure that can implement the healthcare information extraction system of FIG. 1 in accordance to embodiments.

### DETAILED DESCRIPTION

One embodiment is an artificial intelligence (“AI”)/machine learning (“ML”) based tool that extracts healthcare information from the Internet automatically during medical emergencies to aid in clinical decision-making.

Reference will now be made in detail to the embodiments of the present disclosure, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, and circuits have not been described in detail so as not to unnecessarily obscure aspects of the embodiments. Wherever possible, like reference numbers will be used for like elements.

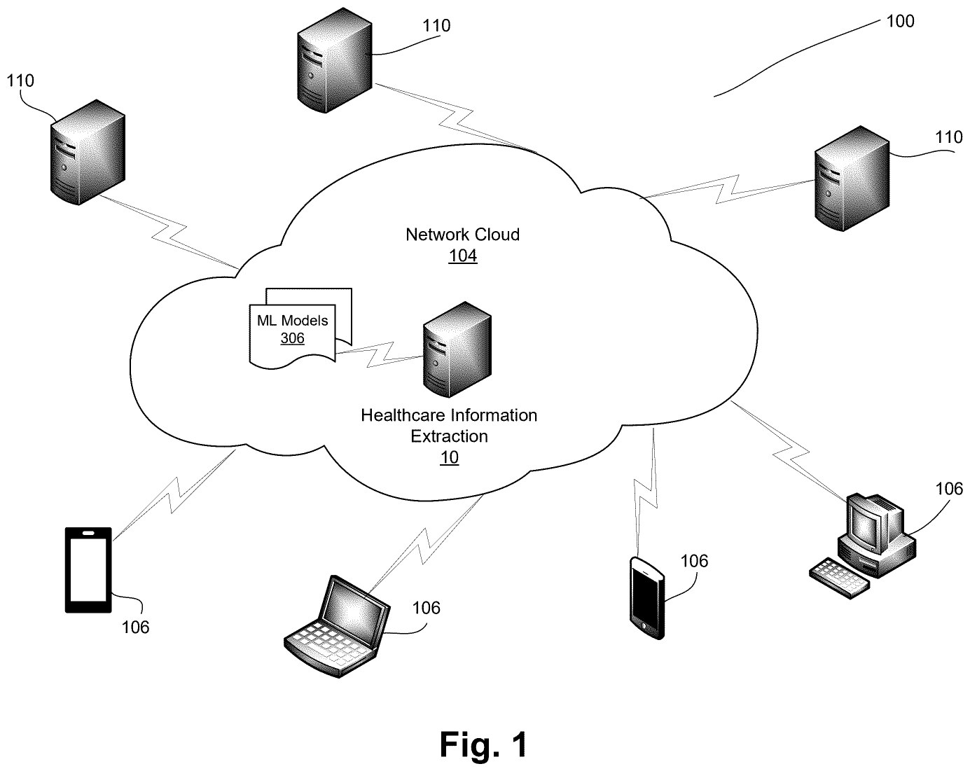

FIG. 1 illustrates an example of a system 100 that includes a healthcare information extraction tool/system 10 in accordance to embodiments. Healthcare information extraction system 10 may be implemented within a computing environment that includes a communication network/cloud 104. Network 104 may be a private network that can communicate with a public network (e.g., the Internet) to access additional services 110 provided by a cloud services provider. Examples of communication networks include a mobile network, a wireless network, a cellular network, a local area network (“LAN”), a wide area network (“WAN”), other wireless communication networks, or combinations of these and other networks. Healthcare information extraction system 10 may be administered by a service provider, such as via the Oracle Cloud Infrastructure (“OCI”) from Oracle Corp.

Tenants of the cloud services provider can be companies or any type of organization or groups whose members include users of services offered by the service provider. Services may include or be provided as access to, without limitation, an application, a resource, a file, a document, data, media, or combinations thereof. Users may have individual accounts with the service provider and organizations may have enterprise accounts with the service provider, where an enterprise account encompasses or aggregates a number of individual user accounts.

System 100 further includes client devices 106, which can be any type of device that can access network 104 and can obtain the benefits of the functionality of healthcare information extraction system 10 of automatically extracting patient/healthcare information. As disclosed herein, a “client” (also disclosed as a “client system” or a “client device”) may be a device or an application executing on a device. System 100 includes a number of different types of client devices 106 that each is able to communicate with network 104.

Executing on cloud 104 (or otherwise in communication with healthcare information extraction system 10) is one or more machine learning (“ML”) models 306. ML models 306 can be integrated with system 10, or remotely located but in communication with system 10.

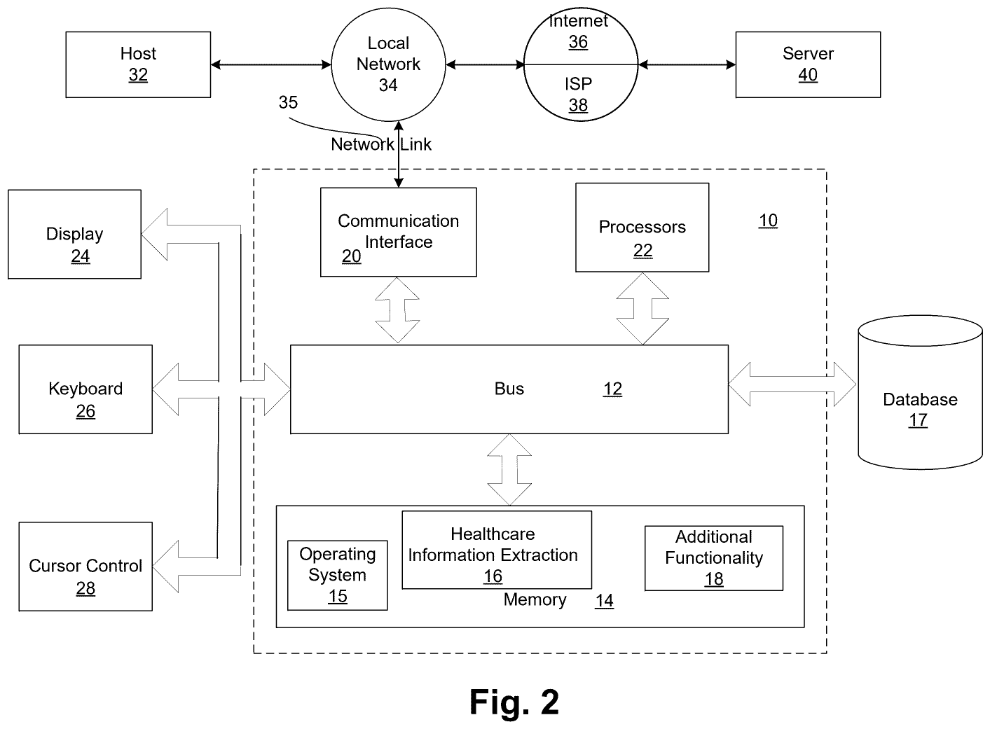

FIG. 2 is a block diagram of healthcare information extraction system 10 of FIG. 1 in the form of a computer server/system 10 in accordance to an embodiment of the present invention. Although shown as a single system, the functionality of system 10 can be implemented as a distributed system. Further, the functionality disclosed herein can be implemented on separate servers or devices that may be coupled together over a network. Further, one or more components of system 10 may not be included. One or more components of FIG. 2 can also be used to implement any of the elements of FIG. 1.

System 10 includes a bus 12 or other communication mechanism for communicating information, and a processor 22 coupled to bus 12 for processing information. Processor 22 may be any type of general or specific purpose processor. System 10 further includes a memory 14 for storing information and instructions to be executed by processor 22. Memory 14 can be comprised of any combination of random access memory (“RAM”), read only memory (“ROM”), static storage such as a magnetic or optical disk, or any other type of computer readable media. System 10 further includes a communication interface 20, such as a network interface card, to provide access to a network. Therefore, a user may interface with system 10 directly, or remotely through a network, or any other method.

Computer readable media may be any available media that can be accessed by processor 22 and includes both volatile and nonvolatile media, transitory and non-transitory media, removable and non-removable media, and communication media. Communication media may include computer readable instructions, data structures, program modules, or other data in a modulated data signal such as a carrier wave or other transport mechanism, and includes any information delivery media.

Processor 22 is further coupled via bus 12 to a display 24, such as a Liquid Crystal Display (“LCD”). A keyboard 26 and a cursor control device 28, such as a computer mouse, are further coupled to bus 12 to enable a user to interface with system 10.

In one embodiment, memory 14 stores software modules that provide functionality when executed by processor 22. The modules include an operating system 15 that provides operating system functionality for system 10. The modules further include a healthcare information extraction module 16 that automatically extracts healthcare/patient information for medical emergencies using AI/ML, and all other functionality disclosed herein. System 10 can be part of a larger system. Therefore, system 10 can include one or more additional functional modules 18, such as an electronic medical records (“EMR”) integrated solution. A file storage device or database 17 is coupled to bus 12 to provide centralized storage for modules 16 and 18, including patient data, historical procedures, physician records, etc. In one embodiment, database 17 is a relational database management system (“RDBMS”) that can use Structured Query Language (“SQL”) to manage the stored data.

In embodiments, communication interface 20 provides a two-way data communication coupling to a network link 35 that is connected to a local network 34. For example, communication interface 20 may be an integrated services digital network (“ISDN”) card, cable modem, satellite modem, or a modem to provide a data communication connection to a corresponding type of telephone line or Ethernet. As another example, communication interface 20 may be a local area network (“LAN”) card to provide a data communication connection to a compatible LAN. Wireless links may also be implemented. In any such implementation, communication interface 20 sends and receives electrical, electromagnetic or optical signals that carry digital data streams representing various types of information.

Network link 35 typically provides data communication through one or more networks to other data devices. For example, network link 35 may provide a connection through local network 34 to a host computer 32 or to data equipment operated by an Internet Service Provider (“ISP”) 38. ISP 38 in turn provides data communication services through the Internet 36. Local network 34 and Internet 36 both use electrical, electromagnetic or optical signals that carry digital data streams. The signals through the various networks and the signals on network link 35 and through communication interface 20, which carry the digital data to and from computer system 10, are example forms of transmission media.

System 10 can send messages and receive data, including program code, through the network(s), network link 35 and communication interface 20. In the Internet example, a server 40 might transmit a requested code for an application program through Internet 36, ISP 38, local network 34 and communication interface 20. The received code may be executed by processor 22 as it is received, and/or stored in database 17, or other non-volatile storage for later execution.

In one embodiment, system 10 is a computing/data processing system including an application or collection of distributed applications for enterprise organizations, and may also implement logistics, manufacturing, and inventory management functionality. The applications and computing system 10 may be configured to operate locally or be implemented as a cloud-based networking system, for example in an infrastructure-as-a-service (“IAAS”), platform-as-a-service (“PAAS”), software-as-a-service (“SAAS”) architecture, or other type of computing solution.

As disclosed, in the emergency room (“ER”) of hospitals, frequently patients are brought in a state of being unconscious, semi-conscious, delirious, stupor, inebriated, etc., and therefore they cannot provide relevant information that helps with clinical decision making. The only clues about the condition of the patient may be information that comes from any identity cards they may be carrying, such as a Social Security card, a driver's license, a school or employee ID card, a library card, etc. These ID cards may give demographic details such as name, age, telephone number etc., but likely do not convey medical information that aids clinical decisions relevant to the present condition.

Therefore, embodiments automatically search the Internet or other publicly available information for all relevant information on the patient that may help in clinical decision making. The searching uses the patient's demographic details as identifiers and yields relevant information from web pages such as social networking sites (e.g., Facebook, Instagram, Twitter), professional networking sites (e.g., LinkedIn), patient's personal websites, employer websites, patient discussion forums, blogging sites, etc., where information is openly available for public viewing. Embodiments extract the information and arrange it in order of high-to-low medical relevance and present it to the ER physician. As a result, embodiments have the potential to reduce morbidity, save precious time in medical emergencies and save lives.

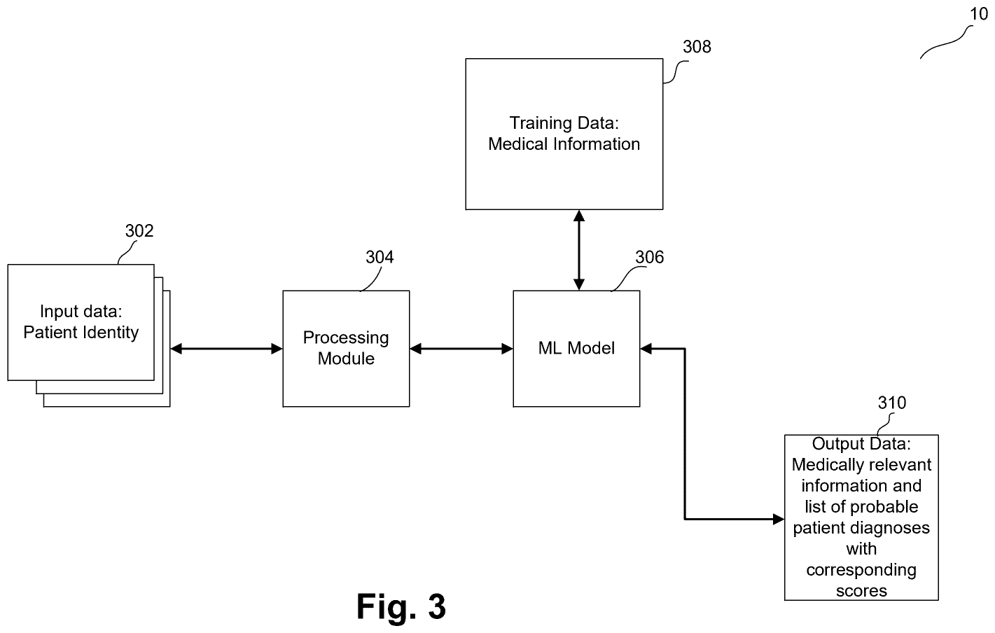

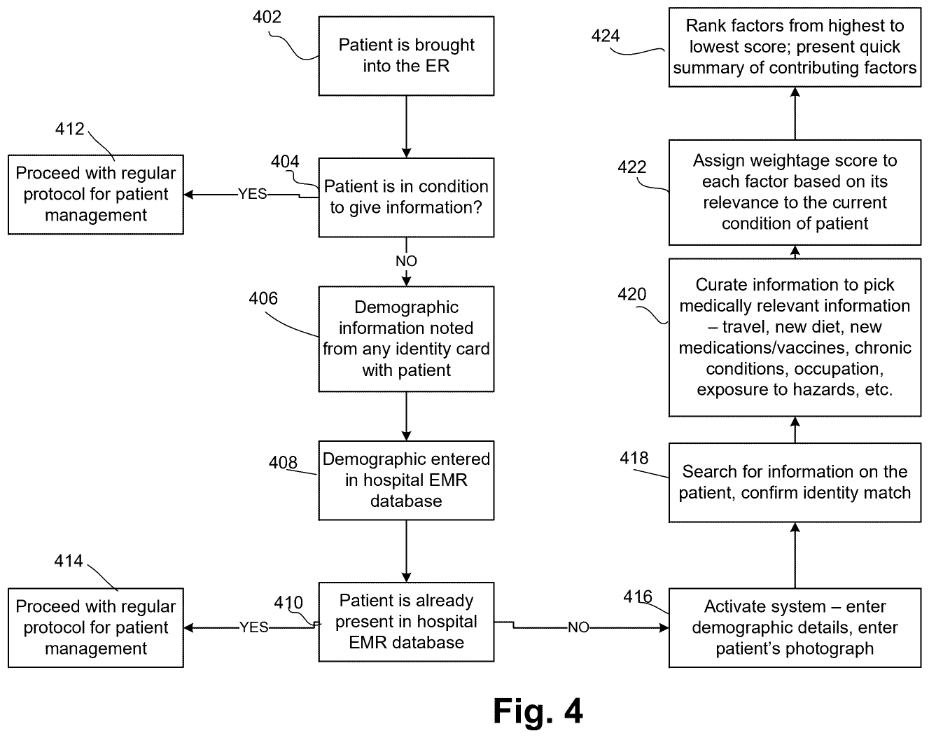

FIG. 3 is a flow/block diagram of the functionality of healthcare information extraction system 10 of FIG. 1 when extracting healthcare/patient information in accordance to embodiments. In one embodiment, the functionality of the flow/block diagram of FIG. 3 (and FIG. 4 below) is implemented by software stored in memory or other computer readable or tangible medium, and executed by a processor. In other embodiments, the functionality may be performed by hardware (e.g., through the use of an application specific integrated circuit (“ASIC”), a programmable gate array (“PGA”), a field programmable gate array (“FPGA”), etc.), or any combination of hardware and software.

System 10 includes input data 302, a processing model 304, an ML model 306, training data 308 and output data 310. In general, system 10 is trained to search the Internet for any medically relevant information regarding the patient in question. Therefore, system 10 in embodiments (1) Identifies medically relevant information on the Internet or other publicly accessible sources; (2) Provides weightage to the above medically relevant information depending on the particular context of the patient; (3) Presents the weighted facts to the medical provider in a reduced order of importance; and (4) Learns (i.e., is retrained) constantly to improve its performance with time.

In connection with weighing/weightage, system 10 is trained to sift through the information it has collected and assign weightage scores based on their relevance to the present condition of the patient. As an example, when a patient is brought into the ER with a high fever and a semi-comatose state, assume system 10 determines from scouring the Internet or other public sources that the patient (1) Was planning to travel to Africa; (2) Started a new hobby of watercolor painting; (3) Had underwent his annual colonoscopy last week; and (4) Was planning on meeting his parents who lived 23 miles away in a Chicago suburb before his travel to Africa.

Among these four pieces of information, the fact that has the most relevancy to his present condition is his travel to Africa. System 10 therefore assigns the highest weightage score to this fact, followed by the colonoscopy (i.e., medical information), and lower scores to his new hobby and his trip to meet his parents, as it probably has least relevance to the patient's present condition because they have the least probability of causation/correlation with his present condition. Specifically, system 10 “knows” from its medical training that his new hobby and visit to parents has the least likelihood of having connected events that could have led to his present condition. It assigns a probability to these, and then finds that the highest probability is with his Africa visit, since medical knowledge dictates that he could have been infected with malaria while in Africa. But water coloring or visiting a Chicago suburb will have the least likelihood of any event happening that could medically lead to his present condition. Therefore, the weightage depends on the probability of likelihood—the higher the probability, higher the weightage.

Training data 308 allows model 306 to identify medically relevant information, and in general includes concepts of medicine. After training, the testing of model 306's understanding of medical information can be done using any of the standardized medical examinations used for training physicians in medical schools. Model 306 is trained to search for, identify and fetch medically relevant information about the patient that aids the ER physician in arriving at a diagnosis, and can also predict possible diagnoses.

Once system 10 is able to achieve a desired level of understanding of medical knowledge, it can be trained to gather such information about any given individual from the Internet. It could use a variety of Internet sources such as social media sites, blogs, forums, corporate pages, video repositories (e.g., YouTube), institutional pages, publications, news media, etc., that are openly accessible to the public. These sources can correspond to the patient, or correspond to someone else but also provide relevant information on the patient (e.g., videos generated from someone else that, on purpose, or inadvertently, includes video of the patient).

System 10 is also trained to pick up information that may not look medically relevant at first glance but may lead to clues regarding the patient. For example, if the patient had mentioned in their blog that they would be traveling to Africa for 3 weeks, it is possible that the present comatose and febrile condition of the patient could be due to cerebral malaria, which they were infected with while in Africa. Therefore, model 306 is implemented with AI algorithms that “connect the dots” between seemingly unconnected facts to deduce medical information and clues to the present condition of the patient.

Training data 308 may be labeled data. Processing module 304 can be used to process input data 302 (i.e., “live” patient identity data).

ML model 306 can be any type of machine learning model (e.g., generative model, neural network, deep learning, NLP, support vector machine (“SVM”), random forests, gradient boosting, large language model (“LLM”) etc.) that is trained by training data 308. In one embodiment, ML model 306 implements generative AI. Generative AI refers to artificial intelligence systems that can create new content, such as text, images, music, and other media, by learning from existing data. Unlike traditional AI, which typically focuses on recognizing patterns and making predictions based on input data, generative AI models are designed to generate new, original outputs that are similar to the data they were trained on. ML model 306 generates output data 310, which includes medically relevant information about the patient that aids the ER physician in arriving at a diagnosis, as well as a weighted list of predictions of possible medical diagnoses for the patient.

System 10 constantly updates/trains ML model 306 using new and/or revised training data 308. This training data can include the latest developments in medical research obtained by regularly scouring through medical journals and published medical literature on websites such as PubMed, CDC, WHO, NIH, FDA, as well as websites and information from medical provider facilities such as Mayo Clinic, Mass. General Hospital, Johns Hopkins, Cleveland Clinic, Harvard Medical School, etc.

Further, when predicted weighted results are presented to an ER physician, if the physician still decides to go with a diagnosis that is lower on the list, with a lower weighted score, system 10 can learn from this experience that there is something that made the physician choose the fact that was lower on the list than the one that was predicted to have a higher score. This way system 10 gets iteratively better each time in assigning scores to the information it finds on the Internet.

FIG. 4 is a flow diagram of the functionality of healthcare information extraction system 10 of FIG. 1 when extracting medical/healthcare information for an ER patient in accordance to embodiments.

At 402, a patient is brought into the ER.

At 404, it is determined if the patient is in condition to give information (proceed to 412 with regular protocol for patient management) or if the patient has no relative/friend/caretaker and is not in a condition to give information to the physician (proceed to 406).

At 406, basic demographic information such as name, age, date of birth and address is received from any identity card or other form of information, including a photograph, present in the patient's possession

At 408, the above information at 406 is entered first in the hospital's EMR database to see if the patient is already registered with the hospital.

At 410, if the patient is already present in the EMR database, at 414 proceed with regular protocol for patient management.

If no at 410, at 416 the tool/system 10 is “activated” for the patient, and the demographic details for the patient and/or photograph of the patient is entered and a search is triggered.

At 418, embodiments search for information on the patient in web pages, including social and professional networking sites, blog sites, video uploading sites such as YouTube, patient forums, etc., and tries to match the demographic information of patient, including their facial photograph, with any photograph available on the above sites to ensure identity match. After ensuring that information available on the Internet about the patient matches the identity of the patient, embodiments start collecting information that may be relevant to the current state of the patient.

At 420, embodiments collect information about the patient which the patient themselves may have revealed, such as their travel (especially to tropical countries which carry risk of certain illnesses), a new kind of diet they may have started, any new medications/vaccinations and their adverse effects, any specific-condition/rare disease patient forums they may be in, any prior chronic conditions they may be having, their place of work/employer, any potentially harmful exposure to chemicals/radiation/pathogens (due to travel/work), etc. Information is curated to pick medically relevant information—travel, new diet, new medications/vaccines, chronic conditions, occupation, exposure to hazards, etc.

At 422, embodiments assign a score to each factor in terms of its weightage in contributing to the probability of the present patient condition.

At 424, embodiments then rank factors according to their score and present a quick summary, with potential differential diagnoses, thus enabling medical personnel to pay attention to the most likely contributing factor, so the right clinical decision is made.

One embodiment utilizes natural language processing (“NLP”) when searching web pages. Information about the patient that is extracted from the web is systematically organized under specific headings/categories such as: travel related information, prior chronic conditions, occupation related information, etc.

Embodiments are configured to bring in information from disparate sources, organize them and rank them based on relevance to the present condition.

As an example, in the case of an unconscious patient—if the travel details gathered from the web show that he travelled to tropical Congo 2 weeks ago, then returned to US and traveled to Hawaii 4 days ago, embodiments will give higher weightage to “travel to Congo”, with a diagnosis of Cerebral Malaria as a potential cause due to its incubation period of 10-14 days.

As another example, in the case of a semi-conscious patient—if the details from publicly available information show that the patient is a software engineer who does long-distance running as a hobby, embodiments give higher weightage to running, with a potential differential diagnosis of dehydration/electrolyte imbalance induced semi-consciousness.

As another example, in the case of a comatose patient—embodiments determine from a diabetics forum on the web that the patient had mentioned that he has been asked to take a higher dose of insulin for his diabetes. Embodiments consider insulin overdose leading to hypoglycemia as a potential cause and gives higher weightage to it.

Embodiments are configured to self-ingest medical literature (i.e., training data 308) using technologies such as cognitive computing. Cognitive computing refers to advanced computing systems that simulate human thought processes to help solve complex problems. It combines technologies from artificial intelligence (“AI”), machine learning, natural language processing (“NLP”), neural networks, and data analytics to mimic how the human brain functions. This enables embodiments to keep itself updated on latest developments in medicine. Embodiments use this information to arrive at potential differential diagnoses from information available on the patient from the Internet.

In connection with 420 of FIG. 4, medically relevant information includes any or all information that may have a bearing on one's health. Other than actual medical information, such as chronic conditions, vaccinations, medications, etc., this includes (but not restricted to) one's location of work and living, lifestyle, environment, diet, activities, hobbies and travel. Thus, multiple factors under the above topics can be relevant medically.

Therefore, as a first step, embodiments gather whatever information is available regarding the patient on the Internet or any other publicly available sources. Then it curates this information to pick only those data points that fall under the above topics, and discards any other information.

In embodiments, in order to determine which weightages to assign for each factor at 422 of FIG. 4, embodiments are fed with basic medical information, and in addition it automatically assimilates medical information from medical literature using technologies such as cognitive computing. This enables embodiments to understand which of the above factors has a greater bearing (weightage) for the current patient's condition.

For example, if a patient is brought unconscious to the ER, and embodiments finds the following information about the patient on the Internet:
1. Two weeks ago, the patient visited Niagara Falls and posted photos;
2. Three days ago, he revealed in his blog that he had been diagnosed with Type II diabetes;
3. One day ago, he posted photos with his pet dog.
   Embodiments assign the highest weightage/score to fact number 2 (recent diagnosis of diabetes), since it is possible that overdose of a hypoglycemic drug may have caused low glucose and loss of consciousness. Embodiments assign lower scores to fact numbers 1 and 3.

Embodiments implement AI/ML for several functions. Embodiments learn from the weightages it gives factors, by comparing them with what decisions are actually taken by the doctor. For example, it may have given the highest score to factor A based on its association with the given condition of the patient. However, if the doctor chooses to go with factor B as the top factor (based on his clinical experience), embodiments learn this. The next time, it gives a higher score to factor B (or similar factor). In this way, AI/ML is used not only for carrying out its functions, but also for continuous learning and improvement of embodiments.

In connection with 418, information searched by embodiments can include professional and career-related sources such as:
LinkedIn: Professional profile, work history, skills, posts;
Company websites: Bios, press releases, staff directories;
ResearchGate/Academia.edu/Google Scholar: For publications/citations;
Github/Stack Overflow: For projects, contributions, discussions for tech professionals;
Portfolio sites: Personal websites, Behance, Dribble etc.

In connection with 418, information searched by embodiments can include social media profiles such as:
Facebook: Posts, photos, groups, comments;
Instagram: Images, lifestyle posts, stories;
Twitter/X: Public tweets, interactions, followers;
Youtube/Vimeo: Videos, channels, comments;
Reddit: Post and comments (if identifiable username is known);
TikTok: Videos, interactions.

In connection with 418, information searched by embodiments can include public records and government data such as:
Registered voter database (country-specific);
Court case databases: Legal filings (e.g., PACER in the US);
Government gazettes: Appointments, name changes, legal notices;
Professional licenses: Bar councils, medical councils, etc.;
Company registrars: Director information, ownership.

In connection with 418, information searched by embodiments can include media and news sources such as:
Google news/Bing news: Mentions in articles/interviews;
Local newspapers/archives: Older or regional references;
Press release aggregators.

In connection with 418, information searched by embodiments can include search engines and aggregators such as:
Google/Bing;
PipI/Spokeo/PeekYou/BeenVerified;
Whitepages/TrueCaller.

In connection with 418, information searched by embodiments can include online communities and forums such as:
Quora: Answers, questions, biography etc.;
Medium/Substack/Blogs: Personal writings, articles;
Online course platforms.

In connection with 418, information searched by embodiments can include images and reverse search such as:
Google reverse image search and TinEye: Match profile photos with other appearances online.

In connection with 418, information searched by embodiments can include archived pages such as:
Wayback Machine.

### Example Cloud Infrastructure

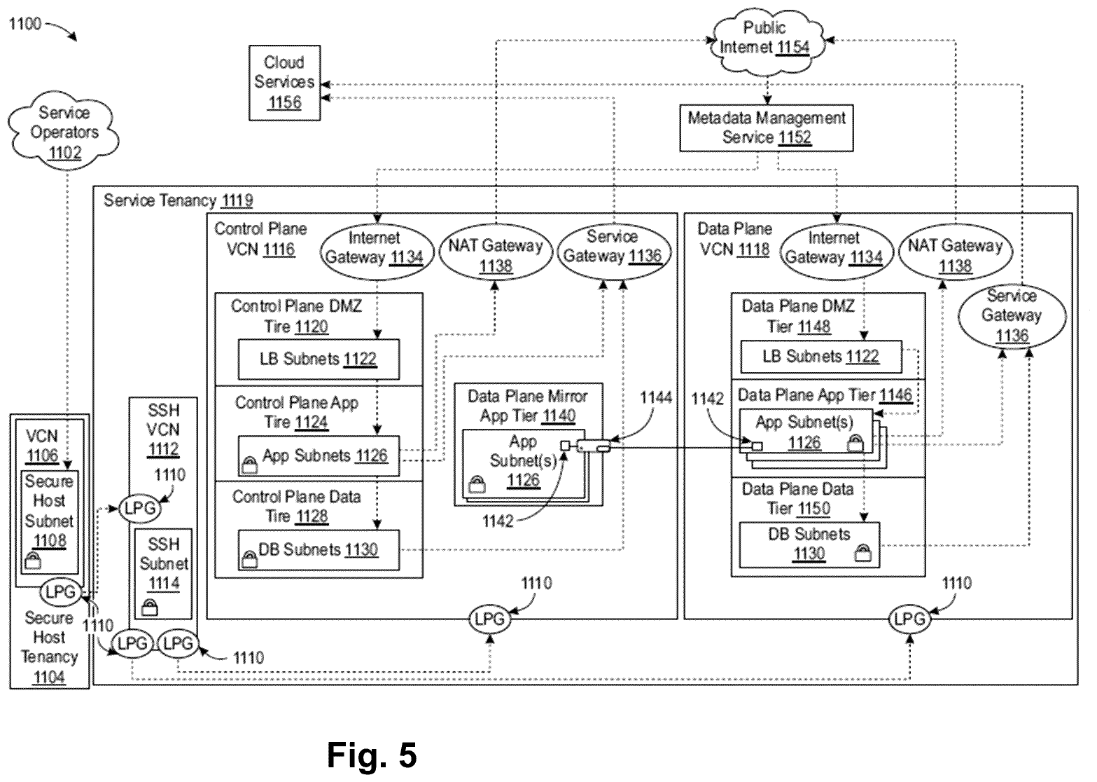

FIGS. 5-8 illustrate an example cloud infrastructure that can implement system 100 that can include healthcare information extraction system 10 of FIG. 1 in accordance to embodiments. The use of the cloud infrastructure, as opposed to an on-premise implementation, allows for training data 308 to be receive from many different users and sources that are interacting with the application of interest, which enhances the accuracy of ML model 306.

As disclosed above, infrastructure as a service (“IaaS”) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (e.g., billing, monitoring, logging, security, load balancing and clustering, etc.). Thus, as these services may be policy-driven, IaaS users may be able to implement policies to drive load balancing to maintain application availability and performance.

In some instances, IaaS customers may access resources and services through a wide area network (“WAN”), such as the Internet, and can use the cloud provider's services to install the remaining elements of an application stack. For example, the user can log in to the IaaS platform to create virtual machines (“VM”s), install operating systems (“OS”s) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different problems for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (“VPC”s) (e.g., a potentially on-demand pool of configurable and/or shared computing resources), also known as a core network. In some examples, there may also be one or more security group rules provisioned to define how the security of the network will be set up and one or more virtual machines. Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 5 is a block diagram 1100 illustrating an example pattern of an IaaS architecture, according to at least one embodiment. Service operators 1102 can be communicatively coupled to a secure host tenancy 1104 that can include a virtual cloud network (“VCN”) 1106 and a secure host subnet 1108. In some examples, the service operators 1102 may be using one or more client computing devices, which may be portable handheld devices (e.g., an iPhone®, cellular telephone, an iPad®, computing tablet, a personal digital assistant (“PDA”)) or wearable devices (e.g., a Meta Quest® head mounted display), running software such as Microsoft Windows Mobile®, and/or a variety of mobile operating systems such as iOS, Windows Phone, Android, BlackBerry 8, Palm OS, and the like, and being Internet, e-mail, short message service (“SMS”), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1106 and/or the Internet.

The VCN 1106 can include a local peering gateway (“LPG”) 1110 that can be communicatively coupled to a secure shell (“SSH”) VCN 1112 via an LPG 1110 contained in the SSH VCN 1112. The SSH VCN 1112 can include an SSH subnet 1114, and the SSH VCN 1112 can be communicatively coupled to a control plane VCN 1116 via the LPG 1110 contained in the control plane VCN 1116. Also, the SSH VCN 1112 can be communicatively coupled to a data plane VCN 1118 via an LPG 1110. The control plane VCN 1116 and the data plane VCN 1118 can be contained in a service tenancy 1119 that can be owned and/or operated by the IaaS provider.

The control plane VCN 1116 can include a control plane demilitarized zone (“DMZ”) tier 1120 that acts as a perimeter network (e.g., portions of a corporate network between the corporate intranet and external networks). The DMZ-based servers may have restricted responsibilities and help keep security breaches contained. Additionally, the DMZ tier 1120 can include one or more load balancer (“LB”) subnet(s) 1122, a control plane app tier 1124 that can include app subnet(s) 1126, a control plane data tier 1128 that can include database (DB) subnet(s) 1130 (e.g., frontend DB subnet(s) and/or backend DB subnet(s)). The LB subnet(s) 1122 contained in the control plane DMZ tier 1120 can be communicatively coupled to the app subnet(s) 1126 contained in the control plane app tier 1124 and an Internet gateway 1134 that can be contained in the control plane VCN 1116, and the app subnet(s) 1126 can be communicatively coupled to the DB subnet(s) 1130 contained in the control plane data tier 1128 and a service gateway 1136 and a network address translation (NAT) gateway 1138. The control plane VCN 1116 can include the service gateway 1136 and the NAT gateway 1138.

The control plane VCN 1116 can include a data plane mirror app tier 1140 that can include app subnet(s) 1126. The app subnet(s) 1126 contained in the data plane mirror app tier 1140 can include a virtual network interface controller (VNIC) 1142 that can execute a compute instance 1144. The compute instance 1144 can communicatively couple the app subnet(s) 1126 of the data plane mirror app tier 1140 to app subnet(s) 1126 that can be contained in a data plane app tier 1146.

The data plane VCN 1118 can include the data plane app tier 1146, a data plane DMZ tier 1148, and a data plane data tier 1150. The data plane DMZ tier 1148 can include LB subnet(s) 1122 that can be communicatively coupled to the app subnet(s) 1126 of the data plane app tier 1146 and the Internet gateway 1134 of the data plane VCN 1118. The app subnet(s) 1126 can be communicatively coupled to the service gateway 1136 of the data plane VCN 1118 and the NAT gateway 1138 of the data plane VCN 1118. The data plane data tier 1150 can also include the DB subnet(s) 1130 that can be communicatively coupled to the app subnet(s) 1126 of the data plane app tier 1146.

The Internet gateway 1134 of the control plane VCN 1116 and of the data plane VCN 1118 can be communicatively coupled to a metadata management service 1152 that can be communicatively coupled to public Internet 1154. Public Internet 1154 can be communicatively coupled to the NAT gateway 1138 of the control plane VCN 1116 and of the data plane VCN 1118. The service gateway 1136 of the control plane VCN 1116 and of the data plane VCN 1118 can be communicatively coupled to cloud services 1156.

In some examples, the service gateway 1136 of the control plane VCN 1116 or of the data plane VCN 1118 can make application programming interface (“API”) calls to cloud services 1156 without going through public Internet 1154. The API calls to cloud services 1156 from the service gateway 1136 can be one-way: the service gateway 1136 can make API calls to cloud services 1156, and cloud services 1156 can send requested data to the service gateway 1136. But, cloud services 1156 may not initiate API calls to the service gateway 1136.

In some examples, the secure host tenancy 1104 can be directly connected to the service tenancy 1119, which may be otherwise isolated. The secure host subnet 1108 can communicate with the SSH subnet 1114 through an LPG 1110 that may enable two-way communication over an otherwise isolated system. Connecting the secure host subnet 1108 to the SSH subnet 1114 may give the secure host subnet 1108 access to other entities within the service tenancy 1119.

The control plane VCN 1116 may allow users of the service tenancy 1119 to set up or otherwise provision desired resources. Desired resources provisioned in the control plane VCN 1116 may be deployed or otherwise used in the data plane VCN 1118. In some examples, the control plane VCN 1116 can be isolated from the data plane VCN 1118, and the data plane mirror app tier 1140 of the control plane VCN 1116 can communicate with the data plane app tier 1146 of the data plane VCN 1118 via VNICs 1142 that can be contained in the data plane mirror app tier 1140 and the data plane app tier 1146.

In some examples, users of the system, or customers, can make requests, for example create, read, update, or delete (“CRUD”) operations, through public Internet 1154 that can communicate the requests to the metadata management service 1152. The metadata management service 1152 can communicate the request to the control plane VCN 1116 through the Internet gateway 1134. The request can be received by the LB subnet(s) 1122 contained in the control plane DMZ tier 1120. The LB subnet(s) 1122 may determine that the request is valid, and in response to this determination, the LB subnet(s) 1122 can transmit the request to app subnet(s) 1126 contained in the control plane app tier 1124. If the request is validated and requires a call to public Internet 1154, the call to public Internet 1154 may be transmitted to the NAT gateway 1138 that can make the call to public Internet 1154. Memory that may be desired to be stored by the request can be stored in the DB subnet(s) 1130.

In some examples, the data plane mirror app tier 1140 can facilitate direct communication between the control plane VCN 1116 and the data plane VCN 1118. For example, changes, updates, or other suitable modifications to configuration may be desired to be applied to the resources contained in the data plane VCN 1118. Via a VNIC 1142, the control plane VCN 1116 can directly communicate with, and can thereby execute the changes, updates, or other suitable modifications to configuration to, resources contained in the data plane VCN 1118.

In some embodiments, the control plane VCN 1116 and the data plane VCN 1118 can be contained in the service tenancy 1119. In this case, the user, or the customer, of the system may not own or operate either the control plane VCN 1116 or the data plane VCN 1118. Instead, the IaaS provider may own or operate the control plane VCN 1116 and the data plane VCN 1118, both of which may be contained in the service tenancy 1119. This embodiment can enable isolation of networks that may prevent users or customers from interacting with other users', or other customers', resources. Also, this embodiment may allow users or customers of the system to store databases privately without needing to rely on public Internet 1154, which may not have a desired level of security, for storage.

In other embodiments, the LB subnet(s) 1122 contained in the control plane VCN 1116 can be configured to receive a signal from the service gateway 1136. In this embodiment, the control plane VCN 1116 and the data plane VCN 1118 may be configured to be called by a customer of the IaaS provider without calling public Internet 1154. Customers of the IaaS provider may desire this embodiment since database(s) that the customers use may be controlled by the IaaS provider and may be stored on the service tenancy 1119, which may be isolated from public Internet 1154.

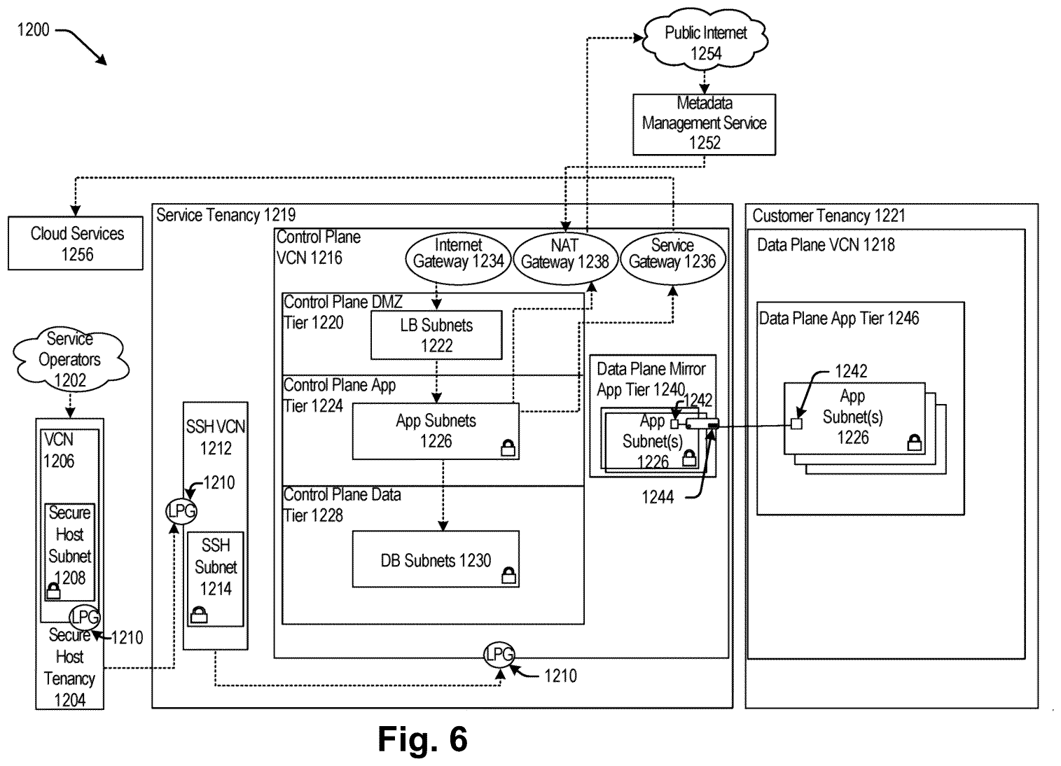

FIG. 6 is a block diagram 1200 illustrating another example pattern of an IaaS architecture, according to at least one embodiment. Service operators 1202 (e.g. service operators 1102) can be communicatively coupled to a secure host tenancy 1204 (e.g. the secure host tenancy 1104) that can include a virtual cloud network (VCN) 1206 (e.g. the VCN 1106) and a secure host subnet 1208 (e.g. the secure host subnet 1108). The VCN 1206 can include a local peering gateway (LPG) 1210 (e.g. the LPG 1110) that can be communicatively coupled to a secure shell (SSH) VCN 1212 (e.g. the SSH VCN 1112 10) via an LPG 1110 contained in the SSH VCN 1212. The SSH VCN 1212 can include an SSH subnet 1214 (e.g. the SSH subnet 1114), and the SSH VCN 1212 can be communicatively coupled to a control plane VCN 1216 (e.g. the control plane VCN 1116) via an LPG 1210 contained in the control plane VCN 1216. The control plane VCN 1216 can be contained in a service tenancy 1219 (e.g. the service tenancy 1119), and the data plane VCN 1218 (e.g. the data plane VCN 1118) can be contained in a customer tenancy 1221 that may be owned or operated by users, or customers, of the system.

The control plane VCN 1216 can include a control plane DMZ tier 1220 (e.g. the control plane DMZ tier 1120) that can include LB subnet(s) 1222 (e.g. LB subnet(s) 1122), a control plane app tier 1224 (e.g. the control plane app tier 1124) that can include app subnet(s) 1226 (e.g. app subnet(s) 1126), a control plane data tier 1228 (e.g. the control plane data tier 1128) that can include database (DB) subnet(s) 1230 (e.g. similar to DB subnet(s) 1130). The LB subnet(s) 1222 contained in the control plane DMZ tier 1220 can be communicatively coupled to the app subnet(s) 1226 contained in the control plane app tier 1224 and an Internet gateway 1234 (e.g. the Internet gateway 1134) that can be contained in the control plane VCN 1216, and the app subnet(s) 1226 can be communicatively coupled to the DB subnet(s) 1230 contained in the control plane data tier 1228 and a service gateway 1236 and a network address translation (NAT) gateway 1238 (e.g. the NAT gateway 1138). The control plane VCN 1216 can include the service gateway 1236 and the NAT gateway 1238.

The control plane VCN 1216 can include a data plane mirror app tier 1240 (e.g. the data plane mirror app tier 1140) that can include app subnet(s) 1226. The app subnet(s) 1226 contained in the data plane mirror app tier 1240 can include a virtual network interface controller (VNIC) 1242 (e.g. the VNIC of 1142) that can execute a compute instance 1244 (e.g. similar to the compute instance 1144). The compute instance 1244 can facilitate communication between the app subnet(s) 1226 of the data plane mirror app tier 1240 and the app subnet(s) 1226 that can be contained in a data plane app tier 1246 (e.g. the data plane app tier 1146) via the VNIC 1242 contained in the data plane mirror app tier 1240 and the VNIC 1242 contained in the data plane app tier 1246.

The Internet gateway 1234 contained in the control plane VCN 1216 can be communicatively coupled to a metadata management service 1252 (e.g. the metadata management service 1152) that can be communicatively coupled to public Internet 1254 (e.g. public Internet 1154). Public Internet 1254 can be communicatively coupled to the NAT gateway 1238 contained in the control plane VCN 1216. The service gateway 1236 contained in the control plane VCN 1216 can be communicatively couple to cloud services 1256 (e.g. cloud services 1156).

In some examples, the data plane VCN 1218 can be contained in the customer tenancy 1221. In this case, the IaaS provider may provide the control plane VCN 1216 for each customer, and the IaaS provider may, for each customer, set up a unique compute instance 1244 that is contained in the service tenancy 1219. Each compute instance 1244 may allow communication between the control plane VCN 1216, contained in the service tenancy 1219, and the data plane VCN 1218 that is contained in the customer tenancy 1221. The compute instance 1244 may allow resources that are provisioned in the control plane VCN 1216 that is contained in the service tenancy 1219, to be deployed or otherwise used in the data plane VCN 1218 that is contained in the customer tenancy 1221.

In other examples, the customer of the IaaS provider may have databases that live in the customer tenancy 1221. In this example, the control plane VCN 1216 can include the data plane mirror app tier 1240 that can include app subnet(s) 1226. The data plane mirror app tier 1240 can reside in the data plane VCN 1218, but the data plane mirror app tier 1240 may not live in the data plane VCN 1218. That is, the data plane mirror app tier 1240 may have access to the customer tenancy 1221, but the data plane mirror app tier 1240 may not exist in the data plane VCN 1218 or be owned or operated by the customer of the IaaS provider. The data plane mirror app tier 1240 may be configured to make calls to the data plane VCN 1218, but may not be configured to make calls to any entity contained in the control plane VCN 1216. The customer may desire to deploy or otherwise use resources in the data plane VCN 1218 that are provisioned in the control plane VCN 1216, and the data plane mirror app tier 1240 can facilitate the desired deployment, or other usage of resources, of the customer.

In some embodiments, the customer of the IaaS provider can apply filters to the data plane VCN 1218. In this embodiment, the customer can determine what the data plane VCN 1218 can access, and the customer may restrict access to public Internet 1254 from the data plane VCN 1218. The IaaS provider may not be able to apply filters or otherwise control access of the data plane VCN 1218 to any outside networks or databases. Applying filters and controls by the customer onto the data plane VCN 1218, contained in the customer tenancy 1221, can help isolate the data plane VCN 1218 from other customers and from public Internet 1254.

In some embodiments, cloud services 1256 can be called by the service gateway 1236 to access services that may not exist on public Internet 1254, on the control plane VCN 1216, or on the data plane VCN 1218. The connection between cloud services 1256 and the control plane VCN 1216 or the data plane VCN 1218 may not be live or continuous. Cloud services 1256 may exist on a different network owned or operated by the IaaS provider. Cloud services 1256 may be configured to receive calls from the service gateway 1236 and may be configured to not receive calls from public Internet 1254. Some cloud services 1256 may be isolated from other cloud services 1256, and the control plane VCN 1216 may be isolated from cloud services 1256 that may not be in the same region as the control plane VCN 1216. For example, the control plane VCN 1216 may be located in “Region 1,” and cloud service “Deployment 8, “may be located in Region 1 and in “Region 2.” If a call to Deployment 8 is made by the service gateway 1236 contained in the control plane VCN 1216 located in Region 1, the call may be transmitted to Deployment 8 in Region 1. In this example, the control plane VCN 1216, or Deployment 8 in Region 1, may not be communicatively coupled to, or otherwise in communication with, Deployment 8 in Region 2.

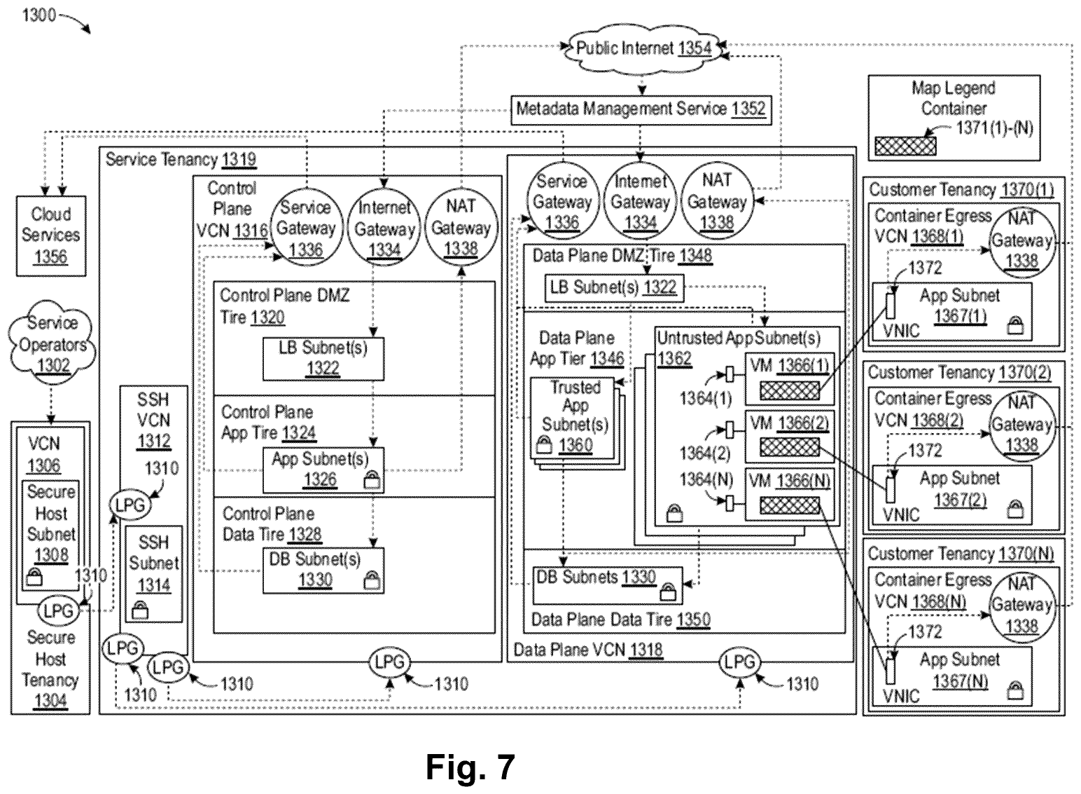

FIG. 7 is a block diagram 1300 illustrating another example pattern of an IaaS architecture, according to at least one embodiment. Service operators 1302 (e.g. service operators 1102) can be communicatively coupled to a secure host tenancy 1304 (e.g., the secure host tenancy 1104) that can include a virtual cloud network (VCN) 1306 (e.g., the VCN 1106) and a secure host subnet 1308 (e.g., the secure host subnet 1108). The VCN 1306 can include an LPG 1310 (e.g., the LPG 1110) that can be communicatively coupled to an SSH VCN 1312 (e.g., the SSH VCN 1112) via an LPG 1310 contained in the SSH VCN 1312. The SSH VCN 1312 can include an SSH subnet 1314 (e.g., the SSH subnet 1114), and the SSH VCN 1312 can be communicatively coupled to a control plane VCN 1316 (e.g., the control plane VCN 1116) via an LPG 1310 contained in the control plane VCN 1316 and to a data plane VCN 1318 (e.g., the data plane 1118) via an LPG 1310 contained in the data plane VCN 1318. The control plane VCN 1316 and the data plane VCN 1318 can be contained in a service tenancy 1319 (e.g., the service tenancy 1119).

The control plane VCN 1316 can include a control plane DMZ tier 1320 (e.g. the control plane DMZ tier 1120) that can include load balancer (“LB”) subnet(s) 1322 (e.g., LB subnet(s) 1122), a control plane app tier 1324 (e.g., the control plane app tier 1124) that can include app subnet(s) 1326 (e.g., similar to app subnet(s) 1126), a control plane data tier 1328 (e.g. the control plane data tier 1128) that can include DB subnet(s) 1330. The LB subnet(s) 1322 contained in the control plane DMZ tier 1320 can be communicatively coupled to the app subnet(s) 1326 contained in the control plane app tier 1324 and to an Internet gateway 1334 (e.g., the Internet gateway 1134) that can be contained in the control plane VCN 1316, and the app subnet(s) 1326 can be communicatively coupled to the DB subnet(s) 1330 contained in the control plane data tier 1328 and to a service gateway 1336 (e.g., the service gateway) and a network address translation (NAT) gateway 1338 (e.g., the NAT gateway 1138). The control plane VCN 1316 can include the service gateway 1336 and the NAT gateway 1338.

The data plane VCN 1318 can include a data plane app tier 1346 (e.g. the data plane app tier 1146), a data plane DMZ tier 1348 (e.g., the data plane DMZ tier 1148), and a data plane data tier 1350 (e.g., the data plane data tier 1150 of FIG. 10). The data plane DMZ tier 1348 can include LB subnet(s) 1322 that can be communicatively coupled to trusted app subnet(s) 1360 and untrusted app subnet(s) 1362 of the data plane app tier 1346 and the Internet gateway 1334 contained in the data plane VCN 1318. The trusted app subnet(s) 1360 can be communicatively coupled to the service gateway 1336 contained in the data plane VCN 1318, the NAT gateway 1338 contained in the data plane VCN 1318, and DB subnet(s) 1330 contained in the data plane data tier 1350. The untrusted app subnet(s) 1362 can be communicatively coupled to the service gateway 1336 contained in the data plane VCN 1318 and DB subnet(s) 1330 contained in the data plane data tier 1350. The data plane data tier 1350 can include DB subnet(s) 1330 that can be communicatively coupled to the service gateway 1336 contained in the data plane VCN 1318.

The untrusted app subnet(s) 1362 can include one or more primary VNICs 1364(1)-(N) that can be communicatively coupled to tenant virtual machines (VMs) 1366(1)-(N). Each tenant VM 1366(1)-(N) can be communicatively coupled to a respective app subnet 1367(1)-(N) that can be contained in respective container egress VCNs 1368(1)-(N) that can be contained in respective customer tenancies 1370(1)-(N). Respective secondary VNICs 1372(1)-(N) can facilitate communication between the untrusted app subnet(s) 1362 contained in the data plane VCN 1318 and the app subnet contained in the container egress VCNs 1368(1)-(N). Each container egress VCNs 1368(1)-(N) can include a NAT gateway 1338 that can be communicatively coupled to public Internet 1354 (e.g. public Internet 1154).

The Internet gateway 1334 contained in the control plane VCN 1316 and contained in the data plane VCN 1318 can be communicatively coupled to a metadata management service 1352 (e.g. the metadata management system 1152) that can be communicatively coupled to public Internet 1354. Public Internet 1354 can be communicatively coupled to the NAT gateway 1338 contained in the control plane VCN 1316 and contained in the data plane VCN 1318. The service gateway 1336 contained in the control plane VCN 1316 and contained in the data plane VCN 1318 can be communicatively couple to cloud services 1356.

In some embodiments, the data plane VCN 1318 can be integrated with customer tenancies 1370. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane tier app 1346. Code to run the function may be executed in the VMs 1366(1)-(N), and the code may not be configured to run anywhere else on the data plane VCN 1318. Each VM 1366(1)-(N) may be connected to one customer tenancy 1370. Respective containers 1371(1)-(N) contained in the VMs 1366(1)-(N) may be configured to run the code. In this case, there can be a dual isolation (e.g., the containers 1371(1)-(N) running code, where the containers 1371(1)-(N) may be contained in at least the VM 1366(1)-(N) that are contained in the untrusted app subnet(s) 1362), which may help prevent incorrect or otherwise undesirable code from damaging the network of the IaaS provider or from damaging a network of a different customer. The containers 1371(1)-(N) may be communicatively coupled to the customer tenancy 1370 and may be configured to transmit or receive data from the customer tenancy 1370. The containers 1371(1)-(N) may not be configured to transmit or receive data from any other entity in the data plane VCN 1318. Upon completion of running the code, the IaaS provider may kill or otherwise dispose of the containers 1371(1)-(N).

In some embodiments, the trusted app subnet(s) 1360 may run code that may be owned or operated by the IaaS provider. In this embodiment, the trusted app subnet(s) 1360 may be communicatively coupled to the DB subnet(s) 1330 and be configured to execute CRUD operations in the DB subnet(s) 1330. The untrusted app subnet(s) 1362 may be communicatively coupled to the DB subnet(s) 1330, but in this embodiment, the untrusted app subnet(s) may be configured to execute read operations in the DB subnet(s) 1330. The containers 1371(1)-(N) that can be contained in the VM 1366(1)-(N) of each customer and that may run code from the customer may not be communicatively coupled with the DB subnet(s) 1330.

In other embodiments, the control plane VCN 1316 and the data plane VCN 1318 may not be directly communicatively coupled. In this embodiment, there may be no direct communication between the control plane VCN 1316 and the data plane VCN 1318. However, communication can occur indirectly through at least one method. An LPG 1310 may be established by the IaaS provider that can facilitate communication between the control plane VCN 1316 and the data plane VCN 1318. In another example, the control plane VCN 1316 or the data plane VCN 1318 can make a call to cloud services 1356 via the service gateway 1336. For example, a call to cloud services 1356 from the control plane VCN 1316 can include a request for a service that can communicate with the data plane VCN 1318.

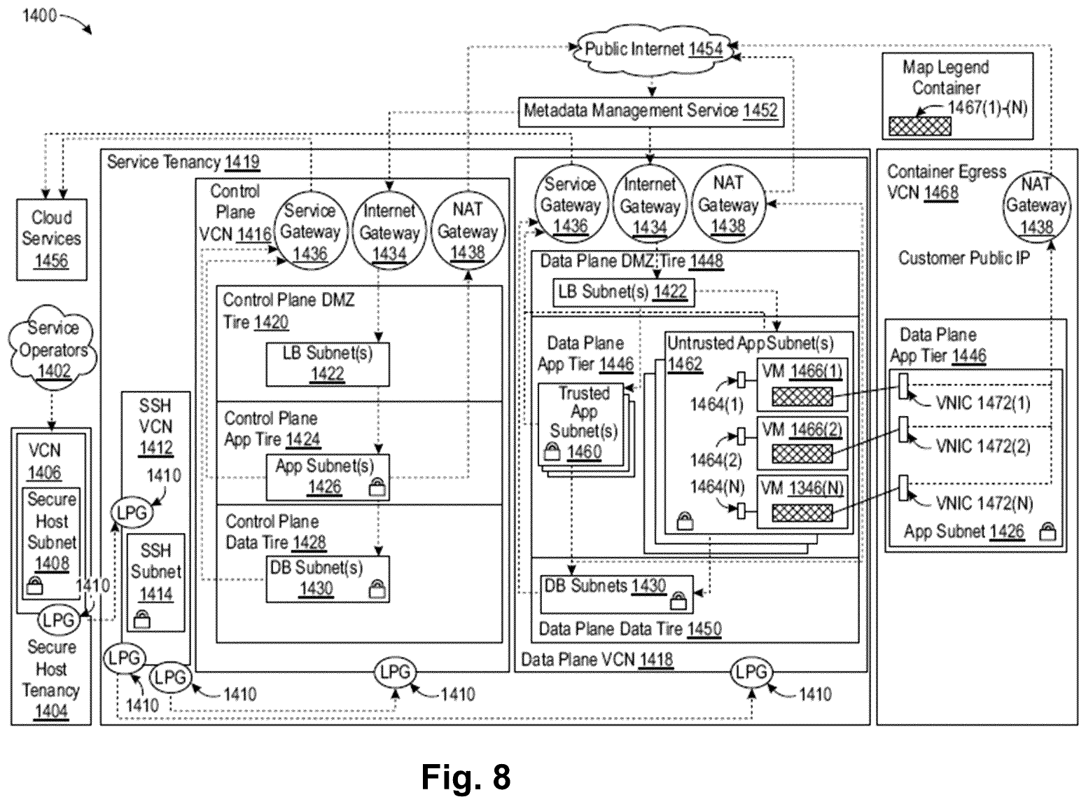

FIG. 8 is a block diagram 1400 illustrating another example pattern of an IaaS architecture, according to at least one embodiment. Service operators 1402 (e.g., service operators 1102) can be communicatively coupled to a secure host tenancy 1404 (e.g., the secure host tenancy 1104) that can include a virtual cloud network (“VCN”) 1406 (e.g., the VCN 1106) and a secure host subnet 1408 (e.g. the secure host subnet 1108). The VCN 1406 can include an LPG 1410 (e.g., the LPG 1110) that can be communicatively coupled to an SSH VCN 1412 (e.g., the SSH VCN 1112) via an LPG 1410 contained in the SSH VCN 1412. The SSH VCN 1412 can include an SSH subnet 1414 (e.g. the SSH subnet 1114), and the SSH VCN 1412 can be communicatively coupled to a control plane VCN 1416 (e.g., the control plane VCN 1116) via an LPG 1410 contained in the control plane VCN 1416 and to a data plane VCN 1418 (e.g., the data plane 1118) via an LPG 1410 contained in the data plane VCN 1418. The control plane VCN 1416 and the data plane VCN 1418 can be contained in a service tenancy 1419 (e.g., the service tenancy 1119).

The control plane VCN 1416 can include a control plane DMZ tier 1420 (e.g., the control plane DMZ tier 1120) that can include LB subnet(s) 1422 (e.g. LB subnet(s) 1122), a control plane app tier 1424 (e.g., the control plane app tier 1124) that can include app subnet(s) 1426 (e.g. app subnet(s) 1126), a control plane data tier 1428 (e.g. the control plane data tier 1128) that can include DB subnet(s) 1430 (e.g., DB subnet(s) 1330). The LB subnet(s) 1422 contained in the control plane DMZ tier 1420 can be communicatively coupled to the app subnet(s) 1426 contained in the control plane app tier 1424 and to an Internet gateway 1434 (e.g. the Internet gateway 1134) that can be contained in the control plane VCN 1416, and the app subnet(s) 1426 can be communicatively coupled to the DB subnet(s) 1430 contained in the control plane data tier 1428 and to a service gateway 1436 (e.g. service gateway 1136) and a network address translation (NAT) gateway 1438 (e.g. NAT gateway 1138). The control plane VCN 1416 can include the service gateway 1436 and the NAT gateway 1438.

The data plane VCN 1418 can include a data plane app tier 1446 (e.g. the data plane app tier 1146), a data plane DMZ tier 1448 (e.g. the data plane DMZ tier 1148), and a data plane data tier 1450 (e.g. the data plane data tier 1150). The data plane DMZ tier 1448 can include LB subnet(s) 1422 that can be communicatively coupled to trusted app subnet(s) 1460 (e.g. trusted app subnet(s) 1360) and untrusted app subnet(s) 1462 (e.g. untrusted app subnet(s) 1362) of the data plane app tier 1446 and the Internet gateway 1434 contained in the data plane VCN 1418. The trusted app subnet(s) 1460 can be communicatively coupled to the service gateway 1436 contained in the data plane VCN 1418, the NAT gateway 1438 contained in the data plane VCN 1418, and DB subnet(s) 1430 contained in the data plane data tier 1450. The untrusted app subnet(s) 1462 can be communicatively coupled to the service gateway 1436 contained in the data plane VCN 1418 and DB subnet(s) 1430 contained in the data plane data tier 1450. The data plane data tier 1450 can include DB subnet(s) 1430 that can be communicatively coupled to the service gateway 1436 contained in the data plane VCN 1418.

The untrusted app subnet(s) 1462 can include primary VNICs 1464(1)-(N) that can be communicatively coupled to tenant virtual machines (VMs) 1466(1)-(N) residing within the untrusted app subnet(s) 1462. Each tenant VM 1466(1)-(N) can run code in a respective container 1467(1)-(N), and be communicatively coupled to an app subnet 1426 that can be contained in a data plane app tier 1446 that can be contained in a container egress VCN 1468. Respective secondary VNICs 1472(1)-(N) can facilitate communication between the untrusted app subnet(s) 1462 contained in the data plane VCN 1418 and the app subnet contained in the container egress VCN 1468. The container egress VCN can include a NAT gateway 1438 that can be communicatively coupled to public Internet 1454 (e.g. public Internet 1154).

The Internet gateway 1434 contained in the control plane VCN 1416 and contained in the data plane VCN 1418 can be communicatively coupled to a metadata management service 1452 (e.g. the metadata management system 1152) that can be communicatively coupled to public Internet 1454. Public Internet 1454 can be communicatively coupled to the NAT gateway 1438 contained in the control plane VCN 1416 and contained in the data plane VCN 1418. The service gateway 1436 contained in the control plane VCN 1416 and contained in the data plane VCN 1418 can be communicatively couple to cloud services 1456.

In some examples, the pattern illustrated by the architecture of block diagram 1400 may be considered an exception to the pattern illustrated by the architecture of block diagram 1300 and may be desirable for a customer of the IaaS provider if the IaaS provider cannot directly communicate with the customer (e.g., a disconnected region). The respective containers 1467(1)-(N) that are contained in the VMs 1466(1)-(N) for each customer can be accessed in real-time by the customer. The containers 1467(1)-(N) may be configured to make calls to respective secondary VNICs 1472(1)-(N) contained in app subnet(s) 1426 of the data plane app tier 1446 that can be contained in the container egress VCN 1468. The secondary VNICs 1472(1)-(N) can transmit the calls to the NAT gateway 1438 that may transmit the calls to public Internet 1454. In this example, the containers 1467(1)-(N) that can be accessed in real-time by the customer can be isolated from the control plane VCN 1416 and can be isolated from other entities contained in the data plane VCN 1418. The containers 1467(1)-(N) may also be isolated from resources from other customers.

In other examples, the customer can use the containers 1467(1)-(N) to call cloud services 1456. In this example, the customer may run code in the containers 1467(1)-(N) that requests a service from cloud services 1456. The containers 1467(1)-(N) can transmit this request to the secondary VNICs 1472(1)-(N) that can transmit the request to the NAT gateway that can transmit the request to public Internet 1454. Public Internet 1454 can transmit the request to LB subnet(s) 1422 contained in the control plane VCN 1416 via the Internet gateway 1434. In response to determining the request is valid, the LB subnet(s) can transmit the request to app subnet(s) 1426 that can transmit the request to cloud services 1456 via the service gateway 1436.

It should be appreciated that IaaS architectures 1100, 1200, 1300, 1400 depicted in the figures may have other components than those depicted. Further, the embodiments shown in the figures are only some examples of a cloud infrastructure system that may incorporate certain embodiments. In some other embodiments, the IaaS systems may have more or fewer components than shown in the figures, may combine two or more components, or may have a different configuration or arrangement of components.

As disclosed, embodiments are directed to a novel method of rapidly extracting medical information about patients from the internet in emergency situations. There are several advantages with embodiments, including: (1) In situations where the patient is not able to convey information to medical personnel that helps in taking the right clinical decisions, an embodiment rapidly searches, collects, filters, ranks, organizes and presents information within seconds/minutes in the ER where time is critically important.

The features, structures, or characteristics of the disclosure described throughout this specification may be combined in any suitable manner in one or more embodiments. For example, the usage of “one embodiment,” “some embodiments,” “certain embodiment,” “certain embodiments,” or other similar language, throughout this specification refers to the fact that a particular feature, structure, or characteristic described in connection with the embodiment may be included in at least one embodiment of the present disclosure. Thus, appearances of the phrases “one embodiment,” “some embodiments,” “a certain embodiment,” “certain embodiments,” or other similar language, throughout this specification do not necessarily all refer to the same group of embodiments, and the described features, structures, or characteristics may be combined in any suitable manner in one or more embodiments.

One having ordinary skill in the art will readily understand that the embodiments as discussed above may be practiced with steps in a different order, and/or with elements in configurations that are different than those which are disclosed. Therefore, although this disclosure considers the outlined embodiments, it would be apparent to those of skill in the art that certain modifications, variations, and alternative constructions would be apparent, while remaining within the spirit and scope of this disclosure. In order to determine the metes and bounds of the disclosure, therefore, reference should be made to the appended claims.

What is claimed is:

1. A method of diagnosing an emergency room (ER) patient, the method comprising:
receiving an identifier of the patient;
searching and retrieving relevant information factors for the patient from publicly available sources using the identifier using a trained machine learning (ML) model, the trained ML model configured to identify and fetch medically relevant information of the patient in response to the identifier of the patient;
weighting each of the retrieved factors relative to contributing to a diagnoses of the patient; and
assigning a score to each of the retrieved factors and providing the scores and corresponding diagnoses to ER personnel.

2. The method of claim 1, further comprising:
training the ML model with medical information and validating the trained machine learning model using standardized medical examinations.

3. The method of claim 1, wherein the publicly available sources comprises at least one of social media sources, blogs, video repositories and publications.

4. The method of claim 1, wherein the identifier comprises at least one of a drivers license, passport, social security number, state identifier, military identifier, alien resident card, or a photograph.

5. The method of claim 1, further comprising:
determining whether the patient is registered in an electronic medical records (EMR) database.

6. The method of claim 1, the trained ML model further configured to predict medical diagnoses in response to the identifier of the patient.

7. The method of claim 2, further comprising:
receiving a selection of one of the diagnoses; and
retraining the ML model based on the selection.

8. The method of claim 1, further comprising:
using a cloud infrastructure for the diagnosing, the cloud infrastructure comprising a first virtual cloud network (VCN) comprising a local peering gateway (LPG) communicatively coupled to a secure shell (SSH) VCN via the LPG;
wherein the LPG is contained in a control plane VCN and the SSH VCN is communicatively coupled to a data plane VCN.

9. A computer readable medium having instructions stored thereon that, when executed by one or more processors, cause the processors to diagnose an emergency room (ER) patient, the diagnosing comprising:
receiving an identifier of the patient;
searching and retrieving relevant information factors for the patient from publicly available sources using the identifier using a trained machine learning (ML) model, the trained ML model configured to identify and fetch medically relevant information of the patient in response to the identifier of the patient;
weighting each of the retrieved factors relative to contributing to a diagnoses of the patient; and
assigning a score to each of the retrieved factors and providing the scores and corresponding diagnoses to ER personnel.

10. The computer readable medium of claim 9, the diagnosing further comprising:
training the ML model with medical information and validating the trained machine learning model using standardized medical examinations.

11. The computer readable medium of claim 9, wherein the publicly available sources comprises at least one of social media sources, blogs, video repositories and publications.

12. The computer readable medium of claim 9, wherein the identifier comprises at least one of a drivers license, passport, social security number, state identifier, military identifier, alien resident card, or a photograph.

13. The computer readable medium of claim 9, the diagnosing further comprising:
determining whether the patient is registered in an electronic medical records (EMR) database.

14. The computer readable medium of claim 9, the trained ML model further configured to predict medical diagnoses in response to the identifier of the patient.

15. The computer readable medium of claim 10, the diagnosing further comprising:
receiving a selection of one of the diagnoses; and
retraining the ML model based on the selection.

16. The computer readable medium of claim 10, the diagnosing further comprising:
using a cloud infrastructure for the diagnosing, the cloud infrastructure comprising a first virtual cloud network (VCN) comprising a local peering gateway (LPG) communicatively coupled to a secure shell (SSH) VCN via the LPG;
wherein the LPG is contained in a control plane VCN and the SSH VCN is communicatively coupled to a data plane VCN.

17. A cloud based system for diagnosing an emergency room (ER) patient, the system comprising:
a trained machine learning (ML) model;
one or more processors coupled to the trained ML model and configured to:
receive an identifier of the patient;
search and retrieve relevant information factors for the patient from publicly available sources using the identifier using the trained ML model, the trained ML model configured to identify and fetch medically relevant information of the patient in response to the identifier of the patient;
weight each of the retrieved factors relative to contributing to a diagnoses of the patient; and
assign a score to each of the retrieved factors and provide the scores and corresponding diagnoses to ER personnel.

18. The cloud based system of claim 17, the processors further configured to:
train the ML model with medical information and validate the trained machine learning model using standardized medical examinations.

19. The cloud based system of claim 17, wherein the publicly available sources comprises at least one of social media sources, blogs, video repositories and publications.

20. The system of claim 17, wherein the system is executed on a cloud infrastructure, the cloud infrastructure comprising a first virtual cloud network (VCN) comprising a local peering gateway (LPG) communicatively coupled to a secure shell (SSH) VCN via the LPG;
wherein the LPG is contained in a control plane VCN and the SSH VCN is communicatively coupled to a data plane VCN.
